(19) United States
(12) Patent Application Publication
(10) Pub. No.: US 2009/0098663 A1
(43) Pub. Date: Apr. 16, 2009

(54) NOVEL WATER-SOLUBLE NANOCRYSTALS COMPRISING A POLYMERIC COATING REAGENT, AND METHODS OF PREPARING THE SAME

(75) Inventors: Mingyong Han, Singapore (SG); Fuke Wang, Singapore (SG)

Correspondence Address: SEED INTELLECTUAL PROPERTY LAW GROUP PLLC, 701 FIFTH AVE, SUITE 5400, SEATTLE, WA, 98104, US

(73) Assignee: AGENCY FOR SCIENCE, TECHNOLOGY AND RESEARCH, Singapore (SG)

(21) Appl. No.: 11/913,673

(22) PCT Filed: May 4, 2005
(86) PCT No.: PCT/SG2005/000136

§ 371 Date: Aug. 25, 2008

(51) Int. Cl. G01N 33/553; C08K 9/00

(52) U.S. Cl. 436/525; 523/205; 523/206

(57) ABSTRACT

Disclosed is a water soluble nanocrystal comprising a nanocrystal core comprising at least one metal M1 selected from an element of main group II, subgroup VIIA, subgroup VIIIA, subgroup IB, subgroup IIB, main group III or main group IV of the periodic system of the elements (PSE), at least one element A selected from main group V or main group VI of the PSE, a capping reagent attached to the surface of the core of the nanocrystal, and a water soluble polymer covalently coupled with the capping reagent to form a water soluble polymer shell over the nanocrystal core. Also disclosed are compositions comprising such nanocrystals and uses of such nanocrystals.

The invention relates to novel water-soluble nanocrystals and to methods of making the same. The invention also relates to the uses of such nanocrystals, including but not limited to, in various analytical and biomedical applications such as the detection and/or visualization of biological materials or processes, e.g. in tissue or cell imaging, in vitro or in vivo. The present invention also relates to compositions and kits containing such nanocrystals which can be used in the detection of analytes such as nucleic acids, proteins or other biomolecules.

Semiconductor nanocrystals (quantum dots) have been receiving great fundamental and technical interest for their use in a variety of technologies, such as light-emitting devices (Colvin et al, Nature 370, 354-357, 1994; Tessler et al, Science 295, 1506-1508, 2002), lasers (Klimov et al, Science 290, 314-317, 2000), solar cells (Huynh et al, Science 295, 2425-2427, 2002) or as fluorescent biological labels in biochemical research areas such as cell biology. For example, see Bruchez et al, Science, Vol. 281, pages 2013-2015, 2001; Chan & Nie, Science, Vol. 281, pages 2016-2018, 2001; U.S. Pat. No. 6,207,392, summarized in Klarreich, Nature, Vol. 43, pages 450-452, 2001; see also Mitchell, Nature Biotechnology, pages 1013-1017, 2001, and U.S. Pat. Nos. 6,423,551, 6,306,610, and 6,326,144.

The development of sensitive non-isotopic detection systems for use in biological assays has significantly impacted many research and diagnostic areas, such as DNA sequencing, clinical diagnostic assays, and fundamental cellular and molecular biology protocols. Current non-isotopic detection methods are mainly based on organic reporter molecules that undergo color change or are fluorescent, luminescent. Fluorescent labeling of molecules is a standard technique in biology. The labels are often organic dyes that give rise to the usual problems of broad spectral features, short lifetime, photobleaching, and potential toxicity to cells. The recent emerging technology of quantum dots has spawned a new era for development of fluorescent labels using inorganic complexes or particles. These materials offer substantial advantages over organic dyes including large Stocks shift, longer emission half-life, narrow emission peak and minimal photo-bleaching (cf. references cited above).

Over the past decade, much progress has been made in the synthesis and characterization of a wide variety of semiconductor nanocrystals. Recent advances have led to large-scale preparation of relatively monodisperse quantum dots. (Murray et al., J. Am. Chem. Soc., 115, 8706-15, 1993; Bowen Katari et al., J. Phys. Chem. 98, 4109-17, 1994; Hines, et al., J. Phys. Chem. 100, 468-71,1996. Dabbousi, et al., J. Phys. Chem. 101, 9463-9475,1997.)

Further advances in luminescent quantum dot technology have resulted in an enhancement of the fluorescence efficiency and stability of the quantum dots. The remarkable luminescent properties of quantum dots arise from quantum size confinement, which occurs when metal and semiconductor core particles are smaller than their excitation Bohr radii, about 1 to 5 nm. (Alivisatos, Science, 271, 933-37, 1996; Alivisatos, J. Phys. Chem. 100, 13226-39, 1996; Brus, Appl Phys., A53, 465-74, 1991; Wilson et al., Science, 262, 1242-46, 1993.) Recent work has shown that improved luminescence can be achieved by capping a size-tunable lower bandgap core particle with a higher band gap inorganic materials shell. For example, CdSe quantum dots passivated with a ZnS layer are strongly luminescence at room temperature, and their emission wavelength can be tuned from blue to red by changing the particle size. Moreover, the ZnS capping layer passivates surface nonradiative recombination sites and leads to greater stability of the quantum dot. (Dabbousi et al., J. Phys. Chem. B101, 9463-75, 1997. Kortan, et al., J. Am. Chem. Soc. 112, 1327-1332, 1990.)

Despite the progress in luminescent quantum dots technology, the conventional capped luminescent quantum dots are not suitable for biological applications because they are not water-soluble.

In order to overcome this problem, the organic passivating layer of the quantum dots were replaced with water-soluble moieties. However, the resultant quantum dots are not highly luminescent (Zhong et al., J. Am. Chem. Soc. 125, 8589, 2003). Short chain thiols such as 2-mercaptoethanol and 1-thio-glycerol have also been used as stabilizers in the preparation of water-soluble CdTe nanocrystals. (Rogach et al., Ber. Bunsenges. Phys. Chem. 100, 1772, 1996; Rajh et al., J. Phys. Chem. 97, 11999, 1993). In another approach, Coffer et al., describe the use of deoxyribonucleic acid (DNA) as a water soluble capping compound (Coffer, et al., Nanotechnology 3, 69, 1992). In all of these systems, the coated nanocrystals were not stable and photoluminescent properties degraded with time.

In a further study, Spanhel et al. disclosed a Cd(OH)₂-capped CdS sol (Spanhel, et al., J. Am. Chem. Soc. 109, 5649, 1987). However, the colloidal nanocrystals could be prepared only in a very narrow pH range (pH 8-10) and exhibited a narrow fluorescence band at a pH of greater than 10. Such pH dependency greatly limits the usefulness of the material, and in particular, it is not appropriate for use in biological systems.

The PCT publication WO 00/17656 discloses core-shell nanocrystals which are capped with a carboxyl acid or sulfonic acid compound of the formula SH(CH₂)ₙ—COOH and SH(CH₂)ₙ—SO₃H, respectively in order to render the nanocrystals water soluble. Similarly, the PCT application WO 00/29617 and British patent application GB 2342651 describe that organic acids such as mercaptoacetic acid or mercapto-undecanoic acid are attached to the surface of nanocrystals to render them water soluble and suitable for conjugation of biomolecules such as proteins or nucleic acids. GB 2342651 also describes the use of trioctylphosphine as capping material that is supposed to confer water solubility of the nanocrystals.

Another approach is taught in PCT publication WO 00/27365, which reports the use of diaminocarboxylic acids as water-solubilising agents. In this PCT publication, the diamino acids are linked to the nanocrystal core by monovalent capping compounds.

PCT publication WO 00/17655 discloses nanocrystals that are rendered water-soluble by the use of a solubilising agent that has a hydrophilic moiety and a hydrophobic moiety. The solubilising agent attaches to the nanocrystal via the hydrophobic group, whereas the hydrophilic group, such as a carboxylic acid or methacrylic acid, provides for water solubility.

In a further PCT application (WO 02/073155), water soluble semiconductor nanocrystals are described in which various molecules such as trioctylphosphin oxide hydroxamates, derivatives of hydroxamic acid or multidentate complexing agents such as ethylenediamine are directly attached to the surface of a nanocrystal to render them water-soluble. These nanocrystals can then be linked to a protein via EDC. In another approach, the PCT application WO 00/58731 discloses nanocrystals which are used for the analysis of blood cell populations and in which amino-derived polysaccharides having a molecular weight from about 3,000 to about 3,000,000 are linked to the nanocrystals.

U.S. Pat. No. 6,699,723 discloses the use of silane-based compounds as linking agent to facilitate the attachment of biomolecules such as biotin and streptavidin to luminescent nanocrystal probes. US Patent Application No. 2004/0072373 A1 describes a method of biochemical labeling using silane-based compounds. Silane-linked nanoparticles are bonded to template molecules by molecular imprinting, and then polymerized to form a matrix. Thereafter, the template molecules are removed from the matrix. The cavity produced in the matrix due to the removal of the template molecule has properties that can be used for labeling.

Recently, the use of synthetic polymers to stabilize water soluble nanocrystals have been reported. US Patent Application No. 2004/0115817 A1 describes that of amphiphilic, diblock polymers can be attached non covalently via hydrophobic interactions to a nanocrystal, the surface of which is coated with agents such as trioctylphosphine or trioctylphosphine oxide. Similarly, Gao et al. (Nature Biotechnology, Vol. 22, 969-976, August 2004) disclose water soluble semiconductor nanocrystals that are encapsulated with amphiphilic, tri-block copolymers via non covalent hydrophobic interactions.

Despite these developments, there remains a need for luminescent nanocrystals that can be used for detection purposes in biological assays. In this respect, it would be is desirable to have nanocrystals that can be attached to a biomolecule in a manner that preserves the biological activity of the biomolecule. Furthermore, it would be desirable to have water-soluble semiconductor nanocrystals which can be prepared and stored as stable, robust suspensions or solutions in aqueous media. Finally, these water-soluble nanocrystals quantum dots should be capable of energy emission with high quantum efficiencies, and should possess a narrow particle size.

Accordingly, it is an object of the invention to provide nanocrystals that meet the above needs.

This object is solved by the nanocrystals and the processes of producing nanocrystals having the features of the respective independent claims.

In one aspect, the invention is directed to a water soluble nanocrystal comprising:

a nanocrystal core comprising at least one metal M1 selected from an element of subgroup Ib, subgroup IIb, subgroup IVb, subgroup Vb, subgroup VIb, subgroup VIIb, subgroup VIIIb, main group II, main group III or main group IV of the periodic system of the elements (PSE), and

a water-soluble shell surrounding the nanocrystal core, said shell comprising:
a first layer comprising a capping reagent attached to the surface of the core of the nanocrystal, said capping reagent having at least one coupling group,
and a second layer comprising a polymer having at least one coupling moiety covalently coupled to the at least one coupling group of the capping reagent.

The water soluble nanocrystal is obtainable by a method comprising:

reacting a nanocrystal core as defined above with a capping reagent, thereby attaching the capping reagent to the surface of the nanocrystal core and forming a first layer surrounding the nanocrystal core,

and

coupling the capping reagent with a polymer having at least one coupling moiety that is reactive towards the at least one coupling group of the capping reagent, thereby forming a second layer covalently coupled to the first layer and completing the formation of a water soluble shell surrounding the nanocrystal core.

In another aspect, the invention is directed to a water soluble nanocrystal comprising:

a nanocrystal core comprising at least one metal M1 selected from an element of main group II, subgroup VIIA, subgroup VIIIA, subgroup IB, subgroup IIB, main group III or main group IV of the periodic system of the elements (PSE), and at least one element A selected from main group V or main group VI of the PSE, and

a water-soluble shell surrounding the nanocrystal core, said shell comprising:
a first layer comprising a capping reagent attached to the surface of the core of the nanocrystal, said capping reagent having at least one coupling group,
   and a second layer comprising a polymer having at least one coupling moiety covalently coupled to the at least one coupling group of the capping reagent. The water soluble nanocrystal is obtainable by a method comprising:

reacting a nanocrystal core as defined above with a capping reagent, thereby attaching the capping reagent to the surface of the nanocrystal core and forming a first layer surrounding the nanocrystal core,

and

coupling the capping reagent with a polymer having at least at least one coupling moiety that is reactive towards the at least one coupling group of the capping reagent, thereby forming a second layer covalently coupled to the first layer and completing the formation of a water soluble shell surrounding the nanocrystal core.

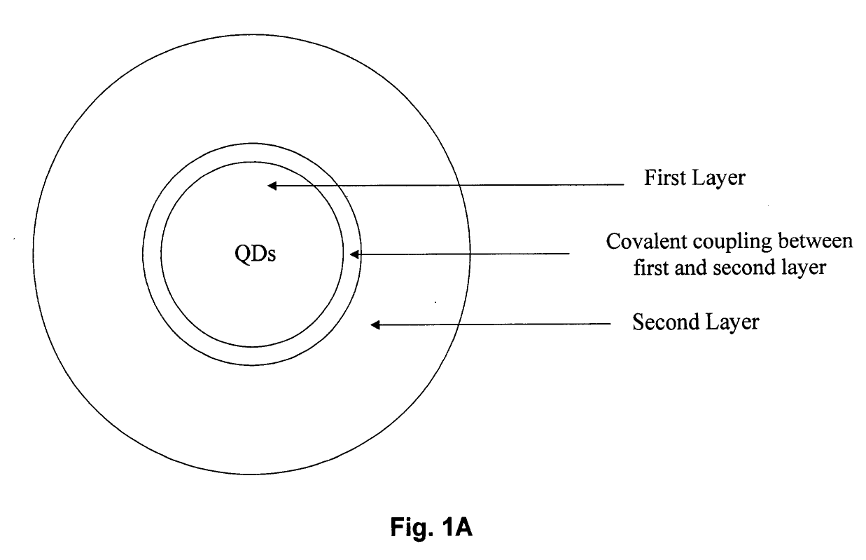

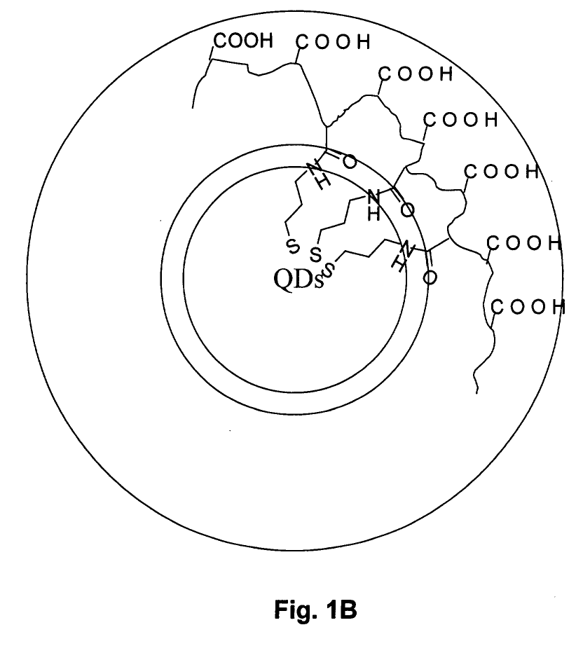

Traditional methods of coating nanocrystals typically do not involve covalent bonding at the interface between the polymer layer and nanocrystals. In the present invention, both small monomers or low molecular weight polymers/oligomers (typically polymers with rather low molecular weight) are first used to cap the nanocrystal surface (for example, to form a metal-sulfur or metal-nitrogen bond) to form a capping reagent layer, also known as the first layer. This first layer is covalently bonded to the nanocrystal core. This step is followed by coupling of a polymer (bearing water soluble groups) to the capping reagent in the presence of a coupling agent. In carrying out the coupling step, the polymer forms a second layer surrounding the nanocrystal core. The polymer may comprise oligomers, polymers, or a mixture thereof. Once the polymer is coupled to the capping reagent, what results is the formation of a water soluble nanocrystal comprising a nanocrystal core surrounded by a water soluble shell (see also FIG. 1).

In another aspect, the invention is directed to a method of preparing a water soluble nanocrystal having a core as defined above comprising:

reacting a nanocrystal core as defined above with a capping reagent, thereby attaching the capping reagent to the surface of the nanocrystal core and forming a first layer surrounding the nanocrystal core,

and

coupling the capping reagent with a polymer having at least at least one coupling moiety that is reactive towards the at least one coupling group of the capping reagent, thereby forming a second layer covalently coupled to the first layer and completing the formation of a water soluble shell surrounding the nanocrystal core.

The present invention is based on the finding that water soluble nanocrystals can be effectively stabilized through the formation of a water soluble polymer shell surrounding the nanocrystal. This shell comprises a first layer (comprising a capping reagent) covalently bonded to the surface of the nanocrystal core, and a second layer (a coating reagent comprising a polymer) which is covalently coupled to the first layer, thereby over-coating the first layer (thus acting as a coating reagent). It is found that a polymer shell synthesized in this manner allows the nanocrystal to stay in an aqueous environment for a reasonably long period of time without any substantial loss of luminescence. Without wishing to be bound by theory, it is believed that the improved stability of the nanocrystals can be attributed to the protective function of the polymer shell. The shell behaves as a hermetic box or protective barrier that reduces contact between the nanocrystal core and reactive water-soluble species such as ions, radicals or molecules that may be present. This is useful for preventing the aggregation of nanocrystals in an aqueous environment. It is thought that in so doing the nanocrystals are kept electrically isolated from each other, thereby also prolonging its photoluminescence. Furthermore, it is also believed that the polymer introduces charges on the surface of the nanocrystal. By having a water soluble polymer shell formed around the nanocrystal, the polymer shell is less readily desorbed from the surface of the nanocrystal as compared to conventional capped nanocrystals. This improves the stability of the nanocrystal in an aqueous environment. On the other hand, small molecules are less suitable as they are more readily desorbed from surface of nanocrystals, thereby exposing the nanocrystal to ionic species that can diffuse through the shell, thereby causing the instability of nanocrystals in aqueous solution. Another advantage is that the (polymer) shell thus formed can also be advantageously functionalized via the attachment of suitable biological molecules or analytes that can facilitate recognition of a huge variety of biological material such as tissues and organ targets. By implementing different combinations of capping reagents and polymers to form the water-soluble shell, the present invention presents an elegant route to a new class of water soluble nanocrystals having improved chemical and physical properties which are useful for a wide variety of applications.

In accordance with the invention, any suitable type of nanocrystal (quantum dot) can be rendered water soluble, so as long as the surface of the nanocrystal can be attached with a capping reagent. In this context, the terms “nanocrystal” and “quantum dot” are used interchangeably.

In one embodiment, suitable nanocrystals have a nanocrystal core comprising metal alone. For this purpose, M1 may be selected from the group consisting of an element of main group II, subgroup VIIA, subgroup VIIIA, subgroup IB, subgroup IIB, main group III or main group IV of the periodic system of the elements (PSE). Accordingly, the nanocrystal core may consist of only the metal element M1; the non-metal element A or B, as defined below, is absent. In this embodiment, the nanocrystal consists only of a pure metal from any of the above groups of the PSE, such as gold, silver, copper (subgroup Ib), titanium (subgroup IVb), terbium (subgroup IIIb), cobalt, platinum, rhodium, ruthenium (subgroup VIIIb), lead (main group IV) or an alloy thereof. While the invention is mainly illustrated in the following with reference only to nanocrystals comprising a counter element A, it is understood that nanocrystals consisting of a pure metal or a mixture of pure metals can also be used in the invention.

In another embodiment, the nanocrystal core used in the present invention may comprise two elements. Accordingly, the nanocrystal core may be a binary nanocrystal alloy comprising two metal elements, M1 and M2, such as any well-known core-shell nanocrystal formed from metals such as Zn, Cd, Hg, Mg, Mn, Ga, In, Al, Fe, Co, Ni, Cu, Ag, Au and Au. Another type of binary nanocrystals suitable in the present invention may comprise one metal element M1, and at least one element A selected from main group V or main group VI of the PSE. Accordingly, the one type of nanocrystal suitable for use presently has the formula M1A. Examples of such nanocrystals may be group II-VI semiconductor nanocrystals (i.e. nanocrystals comprising a metal from main group II or subgroup IIB, and an element from main group VI) wherein the core and/or the shell (the term “shell” as used herein is different and separate from the polymer “shell” made from organic molecules that enclosed the nanocrystal) includes CdS, CdSe, CdTe, MgTe, ZnS, ZnSe, ZnTe, HgS, HgSe, or HgTe. The nanocrystal core may also be any group II-V semiconductor nanocrystal (i.e. nanocrystals comprising a metal from main group III and an element from main group V). The core and/or the shell includes GaN, GaP, GaAs, GaSb, InN, InP, InAs, InSb, AlN, AlP, AlAs, AlSb. Specific examples of core shell nanocrystals that can be used in the present invention include, but are not limited to, (CdSe)-nanocrystals having a ZnS shell, as well as (CdS)-nanocrystals having ZnS shell.

The invention is not limited to the use of the above-described core-shell nanocrystals. In another embodiment, the nanocrystal of the invention can have a core consisting of a homogeneous ternary alloy having the composition M1₁₋ₓM2ₓA, wherein

a) M1 and M2 are independently selected from an element of subgroup IIb, subgroup VIIa, subgroup VIIIa, subgroup Ib or main group II of the periodic system of the elements (PSE), when A represents an element of the main group VI of the PSE, or

b) M1 and M2 are both selected from an element of the main group (III) of the PSE, when A represents an element of the main group (V) of the PSE.

In another embodiment nanocrystal consisting of a homogeneous quaternary alloy can be used. Quaternary alloys of this type have the composition M1₁₋ₓM2ₓA_{y}B_{1-y}, wherein

a) M1 and M2 are independently selected from an element of subgroup IIb, subgroup VIIa, subgroup VIIIa, subgroup Ib or main group II of the periodic system of the elements (PSE), when A and B both represent an element of the main group VI of the PSE, or

b) M1 and M2 are independently selected from an element of the main group (III) of the PSE, when A and B both represent an element of the main group (V) of the PSE.

Examples of this type of homogenous ternary or quaternary nanocrystals have been described, for instance, in Zhong et al, J. Am. Chem. Soc, 2003 125, 8598-8594, Zhong et al, J. Am. Chem. Soc, 2003 125, 13559-13553, or the International patent application WO 2004/054923.

The designation M1 and M2 as used in the formula described above may be used interchangeably throughout the specification. For example, an alloy comprising Cd and Hg can be designated by M1 or M2 as well as M2 and M1, each respectively. Likewise, the designation A and B for elements of group V or VI of the PSE are used interchangeably; thus in an quaternary alloy of the invention Se or Te can both be named as element A or B.

Such ternary nanocrystals are obtainable by a process comprising forming a binary nanocrystal M1A by heating a reaction mixture containing the element M1 in a form suitable for the generation of a nanocrystal to a suitable temperature T1, adding at this temperature the element A in a form suitable for the generation of a nanocrystal, heating the reaction mixture for a sufficient period of time at a temperature suitable for forming said binary nanocrystal M1A and then allowing the reaction mixture to cool, and

reheating the reaction mixture, without precipitating or isolating the formed binary nanocrystal M1A, to a suitable temperature T2, adding to the reaction mixture at this temperature a sufficient quantity of the element M2 in a form suitable for the generation of a nanocrystal, then heating the reaction mixture for a sufficient period of time at a temperature suitable for forming said ternary nanocrystal M1₁₋ₓM2ₓA and then allowing the reaction mixture to cool to room temperature, and isolating the ternary nanocrystal M1₁₋ₓM2ₓA.

In these ternary nanocrystals, the index x has a value of 0.001<x<0.999, preferably of 0.01<x<0.99, 0.1<0.9 or more preferred of 0.5<x<0.95. In even more preferred embodiments, x can have a value between about 0.2 or about 0.3 to about 0.8 or about 0.9. In the quaternary nanocrystals employed here, y has a value of 0.001<y<0.999, preferably of 0.01<y<0.99, or more preferably of 0.1<x<0.95 or between about 0.2 and about 0.8.

In the II-VI ternary nanocrystals, the elements M1 and M2 comprised therein are preferably independently selected from the group consisting of Zn, Cd and Hg. The element A of the group VI of the PSE in these ternary alloys is preferably selected from the group consisting of S, Se and Te. Thus, all combinations of these elements M1, M2 and A are within the scope of the invention. In preferred embodiments nanocrystals used have the composition ZnₓCd₁₋ₓSe, ZnₓCd₁₋ₓS, ZnₓCd₁₋ₓTe, HgₓCd₁₋ₓSe, HgₓCd₁₋ₓTe, HgₓCd₁₋ₓS, ZnₓHg₁₋ₓSe, ZnₓHg₁₋ₓTe, and ZnₓHg₁₋ₓS.

In some preferred embodiments, x as used in the above chemical formulas has a value of 0.10<x<0.90 or 0.15<x<0.85, and more preferably a value of 0.2<x<0.8. In particularly preferred embodiments, the nanocrystals have the composition ZnₓCd₁₋ₓS and ZnₓCd₁₋ₓSe. Such nanocrystals are preferred in which x has a value of 0.10<x<0.95, and more preferably a value of 0.2<x<0.8.

In certain embodiments in which the nanocrystal core is made from III-V nanocrystals of the invention, each of the elements M1 and M2 are independently selected from Ga and In. The element A is preferably selected from P, As and Sb. All possible combinations of these elements M1, M2 and A are within the scope of the invention. In some presently preferred embodiments, nanocrystals have the composition GaₓIn₁₋ₓP, GaₓIn₁₋ₓAs and GaₓIn₁₋ₓAs.

In the invention, the nanocrystal core is encased in a water soluble polymer shell which comprises 2 main components. The first component of the water soluble shell is a capping reagent that has affinity for the surface of the nanocrystal core and that forms the first layer of the polymer shell. The second component is the polymer that is coupled to the capping reagent and which forms the second layer of the water soluble shell

All types of small molecules or macromolecules which have binding affinity to surface of nanomaterials may be used as capping reagents for forming the firs layer. Preferred capping reagents are organic molecules and may have, firstly, at least one moiety that can covalently bond to or be immobilized on the surface of the nanocrystal core, and, secondly, at least one coupling group that provides for subsequent coupling with the polymer. The coupling group may react directly with the coupling moieties present in the polymer, or it may require activation by a coupling agent, for example, in order to proceed with the coupling reaction. Each of these two moieties may be present in the capping reagent either at a terminal location on the molecule, or at a non-terminal location along the main chain of the molecule. Examples of low molecular weight polymers include amino- or carboxyl-rich polymers or mixtures thereof.

In one embodiment, the capping reagent comprises one moiety having affinity for the surface of the core of the nanocrystal, said moiety being located at a terminal position on the capping reagent molecule. The interaction between the nanocrystal core and the moieties may arise from hydrophobic or electrostatic interaction, or from covalent or coordinative bonding. Suitable terminal groups include moieties that have free (unbonded) electron pairs, thereby enabling the capping reagent to be bonded to the surface of the nanocrystal core. Exemplary terminal groups comprise moieties containing S, N, P atoms or a P═O group. Specific examples of these moieties include amine, thiol, amine-oxide and phosphine, for example.

In a further embodiment, the capping reagent further comprises at least one coupling group spaced apart from the terminal group by a hydrophobic region. Each coupling group may comprise any suitable number of main chain carbon atoms, and any suitable functional group that can react with a complementary coupling moiety on the polymer which is used to form the second layer of the water soluble shell. Exemplary coupling moieties may be selected from the group consisting of hydroxy (—OH), amino (—NH₂), carboxyl (—COOH), carbonyl (—CHO), cyano groups (—CN).

In a preferred embodiment, the capping reagent comprises one coupling group which is spaced apart from the terminal group by a hydrophobic region, as illustrated in the following general formula (G1):

TG-HR-CM₁

wherein

TG—terminal group

HR—hydrophobic region

CM₁—coupling group

In a preferred embodiment, the capping reagent comprises two coupling group spaced apart from the terminal group by a hydrophobic region, as illustrated in the following general formula (G2):

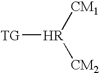

wherein

TG—terminal group

HR—hydrophobic region

CM₁ & CM₂—coupling groups

In the formulas G1 and G2 above, the coupling groups CM1 and CM2 may be hydrophilic. Examples of hydrophilic coupling groups include —NH2, —COOH or OH functional groups. Other examples include nitrile groups, isocyante groups and halides. The coupling groups may also be hydrophobic. A capping reagent having a combination of hydrophobic and hydrophilic groups may be used. Some examples of hydrophobic groups include an alkyl moiety, an aromatic ring, or a methoxy group.

Without wishing to be bound by theory, it is believed that the hydrophobic region in the capping reagent as defined in formula (G1) and (G2) is capable of shielding the nanocrystal core from charged species present in an aqueous environment. Charge transfer from the aqueous environment to the surface of the nanocrystal core becomes hindered by the hydrophobic region, thereby minimizing premature quenching of intermediate nanocrystals (i.e. nanocrystals that are capped with the capping reagent) during synthesis. Thus, the present of the hydrophobic region in the capping reagent can help to improve the final quantum yield of the nanocrystals. Examples of hydrophobic moieties suitable for this purpose include hydrocarbon moieties, including all aliphatic straight-chained, cyclic, or aromatic hydrocarbon moieties.

In one embodiment, the capping reagent used in the nanocrystal of the invention has the general formula (I):

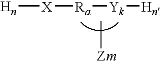

In this formula, X represents a terminal group that has affinity for the surface of the nanocrystal core. X may be selected from S, N, P, or O═P. Specific examples of the moiety Hₙ—X— may include any one of the following: H—S—, O═P—, and H₂N—, for example. Rₐ is a moiety comprising at least 2 main chain carbon atoms, and thus possesses hydrophobic character. If Rₐ is predominantly hydrophobic in character, e.g. a hydrocarbon, it then provides a hydrophobic region separating moiety Z from the nanocrystal core. The moiety Y is selected from N, C, —COO—, or —CH₂O—. Z is a moiety that comprises at least one coupling moiety for subsequent polymerization, and which thus confers a predominantly hydrophilic character to a portion of the hydrophilic capping reagent. Exemplary polar functional groups include, but are not limited to —OH, —COOH, —NH₂, —CHO, —CONHR, —CN, —NCO, —COR and halides. The numerals in the formula are represented by the symbols k, n, n′ and m. k is 0 or 1. The numeral n is an integer from 0 to 3 and n′ is an integer from 0 to 2; both are selected in order to satisfy the valence requirement of X and Y respectively. The numeral m is an integer from 0 to 2. The numeral k is 0 or 1. The condition applies that if k is 0, Z will be bonded to Rₐ. The value of k=0 caters to the case where the coupling moiety Z is directly bonded to Rₐ where, for example, Rₐ is a cyclic moiety, e.g. aliphatic cycloalkanes, aromatic hydrocarbons or heterocycles. However, it is possible that Rₐ is a cyclic moiety when k=1, e.g. a tertiary amino group bonded to a benzene ring, or a cyclic hydrocarbon. Therefore, in the present formula, either Y or Z can function as a coupling group. If Z is present as a coupling group, then Y may function as a structural component for attaching coupling group Z. If Z is absent, Y may then form part of the coupling group.

The moiety Rₐ in the above formula may comprise between several tens to several hundred main chain atoms. In one particular embodiment, each of Rₐ and Z independently comprises 2 to 50 main chain atoms. Z may comprise one or more amide or ester linkages. Examples of suitable moieties which can be used for Rₐ include alkyl, alkenyl, alkoxy and aryl moieties.

The term “alkyl” as used herein refers to a branched or unbranched, straight-chained or cyclic saturated hydrocarbon group, generally comprising 2 to 50 carbon atoms, such as methyl, ethyl, n-propyl, isopropyl, n-butyl, isobutyl, t-butyl, octyl, decyl, tetradecyl, hexadecyl, eicosyl, tetracosyl, as well as cycloalkyl groups such as cyclopentyl, cyclohexyl, for instance. The term “alkenyl” as used herein refers to a branched or unbranched hydrocarbon group generally comprising 2 to 50 carbon atoms and containing at least one double bond, typically containing one to six double bonds, more typically one or two double bonds, e.g. ethenyl, n-propenyl, n-butenyl, octenyl, decenyl, as well as cycloalkenyl groups, such as cyclopentenyl, cyclohexenyl, for instance. The term “alkoxy” as used herein refers to a substituent —O—R wherein R is alkyl as defined above. The term “aryl” as used herein, and unless otherwise specified, refers to an aromatic moiety containing one or more aromatic rings. Aryl groups are optionally substituted with one or more inert, non-hydrogen substituents on the aromatic ring, and suitable substituents include, for example, halo, haloalkyl (preferably halo-substituted lower alkyl), alkyl (preferably lower alkyl), alkenyl (preferably lower alkenyl), alkynyl (preferably lower alkynyl), alkoxy (preferably lower alkoxy), alkoxycarbonyl (preferably lower alkoxycarbonyl), carboxy, nitro, cyano and sulfonyl. In all embodiments, Rₐ may include heteroaromatic moieties, which generally comprise heteroatoms such as nitrogen, oxygen or sulfur.

In a preferred embodiment, Rₐ is selected from the group consisting of ethyl, propyl, butyl and pentyl, cyclopentyl, cyclohexyl, cyclo-octyl, ethoxy, propoxy, butoxy, and benzyl moieties. One embodiment of a preferred capping reagent is selected from the group consisting of aminoethylthiol, aminopropylthiol, and aminobutylthiol.

Examples of some particularly suitable capping reagents are (hydrophilic) compounds having the respective formulas as follows:

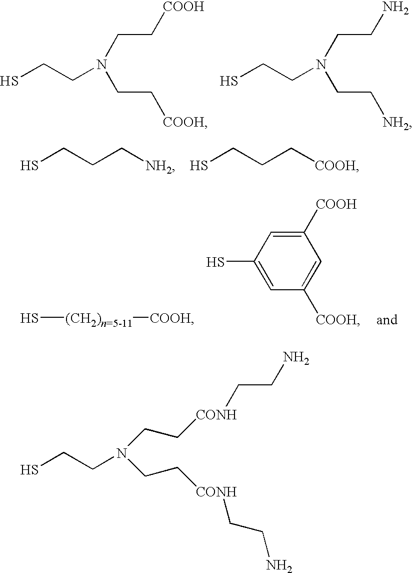

In another embodiment, the capping reagent couples with the polymer via polymerizable unsaturated groups, such as C═C double bonds, via any free radical polymerization mechanism. Specific examples of such capping reagents include, but are not limited to ω-thiol terminated methyl methacrylate, 2-butenethiol, (E)-2-Butene-1-thiol, S-(E)-2-butenyl thioacetate, S-3-methylbutenyl thioacetate, 2-quinolinemethanethiol, and S-2-quinolinemethyl thioacetate

The second component of the water-soluble shell surrounding the nanocrystal core is formed by coupling of a polymer bearing water-soluble groups to the capping reagent, via the use of a coupling agent to activate the coupling groups present in the capping reagent. The coupling agent and the polymer bearing the coupling moieties may be added sequentially, i.e. the polymer is added after the activation has been carried out; or the polymer may be added simultaneously along with the coupling agent.

In principle, any coupling agent that activates the coupling groups in the capping reagent can be used, as long as the coupling agent is chemically compatible with the capping reagent used for forming the first and the polymer used for forming the second layer, meaning that the coupling agent does not react with them to alter their structure. Ideally, no unreacted coupling agent should be present in the nanocrystal as the coupling agent molecules should be completely displaced by polymer molecules. However, in practical reality, it might be possible that unreacted residues of the coupling agent may nevertheless be present in the final nanocrystal.

The determination of an appropriate coupling agent is within the knowledge of the person of average skill in the art. One example of a suitable coupling reagent is 1-ethyl-3-[3-dimethylaminopropyl]carbodiimide (EDC) used in combination with sulfo-N-hydroxysuccinimide (NHS). Other types of coupling reagents may be used, including, but not limited to, imides and azoles. Some examples of imides which can be used are carbodiimides, succinimides and pthalimides. Some explicit examples of imides include 1-ethyl-3-[3-dimethylaminopropyl]carbodiimide (EDC), sulfo-N-hydroxysuccinimide, N,N′-Dicyclohexylcarbodiimide (DCC), N,N′-dicyclohexyl carbodiimide, N-(3-dimethylaminopropyl)-N′-ethylcarbodiimide, used in connection with N-hydroxysuccinimide or any other activation molecule.

In the case of a capping agent in which the coupling group comprises an unsaturated C═C bond, the coupling agent comprises an initiator such as tert-butyl peracetate, tert-butyl peracetate, benzoyl peroxide, potassium persulfate, and peracetic acid.

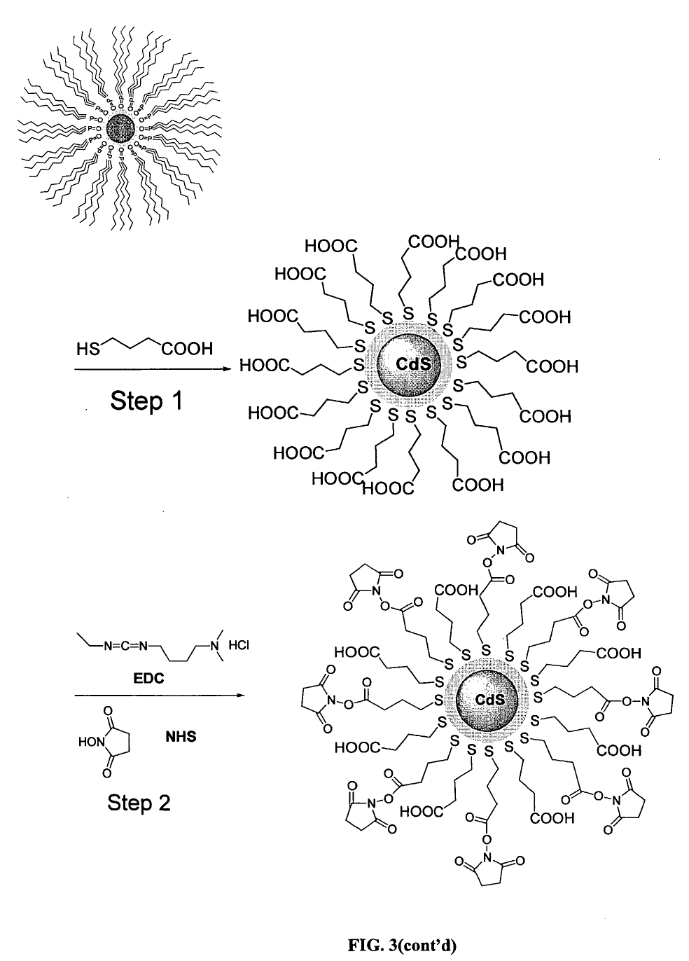

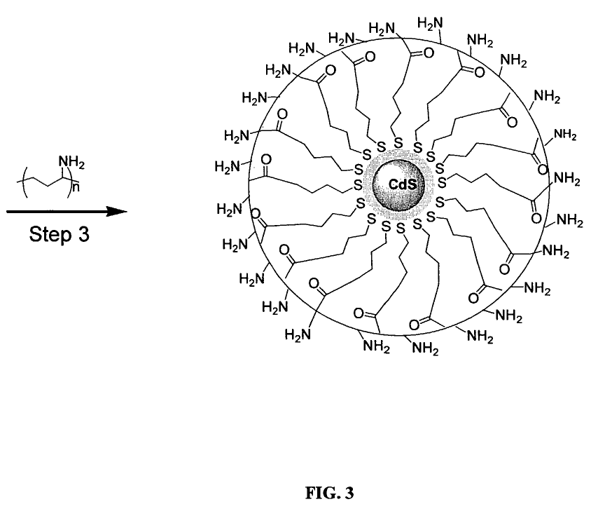

The polymer which is used for forming the second layer of the water-soluble shell may comprise one or more suitable coupling moieties that has coupling moieties which will react with activated coupling groups on the capping reagent. Typically, suitable polymers have coupling moieties that carry 1, 2, 3 or in some embodiments, at least 2 (i.e. a plurality of, functional groups that are reactive towards the activated coupling groups of the capping reagent. As illustrated in FIG. 3, when at least two coupling moieties of the polymer are reacted with molecules of the capping reagent, the polymer becomes covalently coupled (“cross-linked”) to the capping reagent, thereby forming a water soluble polymer shell that surrounds the nanocrystal core.

The coupling of the polymer with the capping reagent can be carried out by means of any suitable coupling reaction scheme. Examples of suitable reaction schemes include free-radical coupling, amide coupling or ester coupling reactions. Apart from using conventional coupling reactions, polymers/oligomers can be grafted onto the capping reagent via suitable coupling reactions, for example. In one embodiment, the polymer to be grafted onto the hydrophilic capping reagent is first synthesized, and then it is coupled to the exposed coupling moieties on the capping reagent via a carbodiimide mediated coupling reaction (i.e. the cross-linking agent). Suitable polymers include random as well as block copolymers bearing functional groups that can be coupled to the hydrophilic capping reagent.

One preferred coupling reaction is the carbodiimide coupling reaction provided by 1-ethyl-3-[3-dimethylaminopropyl]carbodiimide] and promoted by sulfo-N-hydroxysuccinimide, in which carboxyl functional groups and amino functional groups in the coupling groups of the capping reagent and the coupling moieties on the polymer react to form covalent bonds.

In the context of the invention, the term ‘polymer’ that is present as the second layer of the water-soluble shell includes low molecular weight polymers (e.g. oligomer) as well as high molecular weight polymers, ranging from a molecular weight of about 100 to about 1,000,000 Daltons. The lower limit of molecular weight of the polymer may be higher than 100, depending on the size and number of groups present in each repeating unit. If the polymer is derived from a low molecular weight repeating unit (e.g. having small side chains) such as a polyol or a polyamine, then the lower limit of the molecular weight of the polymer can be low. In the case of a polymer in which the repeating units have a high molecular weight (e.g. bearing bulky side chains), then the lower limit may be higher than 100. In some embodiments, the lower limit of molecular weight of a polymer may be about 400, or 500, or 600, or 1000, or 1200, or 1500, or higher at about 2000. The terms “coupling” and “covalent coupling” are used interchangeably to refer generally to any type of reaction which joins two molecules together to form one single, bigger entity, such as the coupling of an acid and an alcohol to form an ester, or the coupling of an acid and an amine to form an amide. Any reaction that can couple the coupling groups and the coupling moieties present in the capping reagent and the polymer are within the meaning of the term. ‘Coupling’ also includes reacting one or more unsaturated groups (e.g. —C═C— double bonds) present as the coupling group in the capping reagent with a corresponding coupling moiety in the polymer in order to covalently bond the polymer to the capping reagent layer.

The polymer may comprise either hydrophilic or hydrophobic moieties, or it may comprise both hydrophilic and hydrophobic moieties, i.e. it is amphiphilic. These moieties may be present in any suitable proportion in the polymer to obtain a desired solubility in the environment in which the nanocrystals of the invention are to be used. For example, in order to improve water solubility of the water soluble shell, the polymer forming the second layer may comprise more hydrophilic moieties than hydrophobic moieties. Conversely, if the shell is to be rendered hydrophobic, a polymer that has a larger number of hydrophobic moieties than hydrophilic moieties may be used.

In one embodiment, the polymer comprising at least one coupling moiety that is reactive towards the coupling group of the capping reagent has the formula (III):

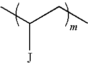

where J is a coupling moiety that is reactive towards the at least one coupling group of the capping reagent, and m is an integer of at least 1.

To illustrate this embodiment, if for instance the first layer has amino-terminated groups, the polymer forming the second layer can have carboxyl groups for covalently coupling with the amino groups of the first layer. In practical, it is possible that not all coupling moieties and coupling groups present are involved in covalent coupling. For example, 50% carboxyl groups may be polymerized with amino groups in the first layer.

In another example, if the first layer have carboxyl-terminated surface, the second layer polymer can have amino groups which covalently couple with the carboxyl groups of the first layer. It is also possible that not all coupling moieties and coupling groups present are involved in covalent coupling. For instance, 50% amino groups may be polymerized with amino groups in the first layer.

In another embodiment, the polymer comprises at least two coupling moieties that are reactive towards the at least one coupling group of the capping reagent. In this case, the polymer may have the formula (IV):

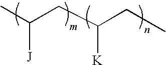

where J and K are coupling moieties, said J and K are the same or different, and each of m and n is an integer of at least 1.

In general, if the capping reagent also has both J and K terminating groups, the polymer can have one or both K and J groups for covalent coupling with the capping reagent. For example, if the first layer has both carboxyl- and amino-terminated surface, the second layer polymer may have only one of or both amino- and carboxyl-groups, respectively, for covalent coupling with the carboxyl groups and amino groups of the first layer. It is sufficient that some of the coupling moieties are covalently coupled to the coupling groups, and it is not necessary for the coupling moieties to be present in exact stoichiometric ratio as the coupling groups.

In yet another embodiment, the polymer comprises at least three coupling moieties that are reactive towards the at least one coupling group of the capping reagent. In this embodiment, said polymer may have the formula (V):

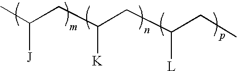

wherein J, K and L are coupling moieties, said J, K and L are the same or different, and each of m, n and p is an integer of at least 1. In a further embodiment, the polymer can have 3 or more different functional groups (NH2, COOH, NCO, CHO, etc) for providing water-solubility as well as surface coupling with the first layer.

The polymer forming the second layer would come into contact with the solvent into which the nanocrystal is placed. Therefore, in order for the nanocrystal to be soluble in the solvent, which may comprise water, for example, at least one of said coupling moieties J, K or L preferably comprises a hydrophilic group which confers water solubility to the water-soluble shell. For this purpose, the polymer may also comprise at least one moiety having a hydrophilic group that confers water solubility to water-soluble shell. The moiety may be present either separately from the coupling moiety or on the coupling moiety itself.

In one embodiment, the coupling moieties J, K and L each comprises a functional group selected from amino, hydroxyl, carbonyl, carboxyl, nitrile, isocyanate and halide groups. If it is desired to have a homofunctional polymer, the coupling moieties of the polymer may be made up solely of, for instance, hydroxyl groups, or carboxyl groups, or amino groups. In such a case, the polymer is, respectively, a polyvinyl alcohol, a polycarboxylic acid, and a polyamine.

In order to obtain nanocrystals with differing properties (e.g. solubility in water), other types of polymers having more than one type of monomer may be used. For example, it is possible to use a diblock copolymer, tri-block copolymer or a mixed random polymer as the polymer for forming the second layer. Specific examples include poly(acrylic acid-b-methyl methacrylate), poly(methyl methacrylate-b-sodium acrylate), poly(t-butyl methacrylate-b-ethylene oxide), poly(methyl methacrylate-b-sodium methacrylate), and poly(methyl methacrylate-b-N,N-dimethyl acrylamide).

The coupling moiety J in the polymer of formula (III) can comprise any suitable functional group that is reactive towards the coupling group present in the capping reagent. The hydrophilic moiety K can comprise any functional group that accords a predominantly hydrophilic character to the polymer, thereby enabling the polymer to be water soluble. Examples of functional groups which are suitable include carboxyl, amino, hydroxyl, amide, ester, anhydride and aldehyde moieties, for example.

In one embodiment, the polymer is selected from the group consisting of a polyamine, a polyacetyl acid, or a polyol. The molecular weight. of the polymer may range from less than about 500 (about 400) to more than about 1,000,000. In one of these embodiments, the molecular weight range may be between about 600 to about 1,400,000, and more preferably between about 2000 to about 750,000. For in vivo applications, the lower limit being of about 2000 may be chosen to minimize the potential toxicity to the human body.

If the capping reagent present comprises polymerizable unsaturated groups as coupling groups, unsaturated polymers can be used for forming the second layer of the water soluble shell, including polyacetylene, polyacrylic acid, polyethylenimine.

In a further embodiment, the polymer may functionalized by attaching an affinity ligand to the polymer. In so doing, a functionalized nanocrystal is obtained. Such a nanocrystal can detect the presence or absence of a substrate for which the affinity ligand has binding specificity. Contact, and subsequent binding, between the affinity ligand of the functionalized nanocrystal and a targeted substrate, if present in the sample, may serve a variety of purposes. For example, it can result in the formation of a complex comprising the functionalized nanocrystal-substrate which can emit a detectable signal for quantization, visualization, or other forms of detection. Contemplated affinity ligands include monoclonal antibodies, including chineric or genetically modified monoclonal antibodies, peptides, aptamers, nucleic acid molecules, streptavidin, avidin, lectin, etc.

In accordance with the above disclosure, another aspect of the present invention concerns a method of preparing a water soluble nanocrystal.

Synthesis of the water-soluble shell can be carried out by first contacting and thereby reacting the capping reagent with the nanocrystal core. The contacting can be done either directly or indirectly. Direct contacting refers to the immersion of the nanocrystal core into a solution containing the capping reagent without the use of any coordinating ligand. Indirect contacting refers the use of a coordinating ligand to prime the nanocrystal core prior to contacting with the capping reagent. Indirect contacting typically comprises two steps. Both methods of contacting are feasible in the present invention. However, the latter method of indirect contacting is preferred as the coordinating ligand helps to speed up the attachment of the capping reagent to the surface of the nanocrystal core.

Indirect contacting will be elaborated as follows. In the first step of indirect contacting, the coordinating ligand is prepared by dissolving in an organic solvent. Next, the nanocrystal core is immersed in the organic solvent for a predetermined period of time, so that a sufficiently stable passivating layer is formed on the surface of the core of the nanocrystal (hereinafter referred to as “passivated nanocrystal”). This passivating layer serves to repel any hydrophilic species which may contact the nanocrystal core, thereby preventing any degradation of the nanocrystal. The passivated nanocrystal can be isolated and stored, if desired, for any desired period of time in the organic solvent containing the coordinating ligand. If desired, a suitable neutral organic solvent, for example, chloroform, methylene chloride, or tetrahydrofuran, may be added.

In the second step of indirect contacting, ligand exchange may be carried out in the presence of an organic solvent or in an aqueous solution. Ligand exchange (displacement) is carried out by adding an excess of the capping reagent to the passivated nanocrystal to facilitate contact of the passivated nanocrystals with the capping reagent. The contact time required to achieve high levels of displacement may be shortened by agitating or sonicating the reaction mixture for a required period of time. After a sufficient length of time, the capping reagent displaces the passivating layer and becomes itself attached to the nanocrystal, thus capping the surface of the nanocrystal core for subsequent coupling of the polymer.

The coordinating ligand used in indirect contacting can be any molecule that comprises a moiety having affinity toward the surface of the nanocrystal core. This affinity can manifest in the form of electrostatic interaction, covalent bonding or coordination bonding, for example. Suitable coordinating ligands include, but are not restricted to, hydrophobic molecules, or amphiphilic molecules comprising a hydrophobic chain attached to a hydrophilic moiety, such as a polar functional group. Examples of such molecules include trioctylphosphine, trioctylphosphine oxide, or mercaptoundecanoic acid. Other types of coordinating ligands that may be used include thiols, amines or silanes.

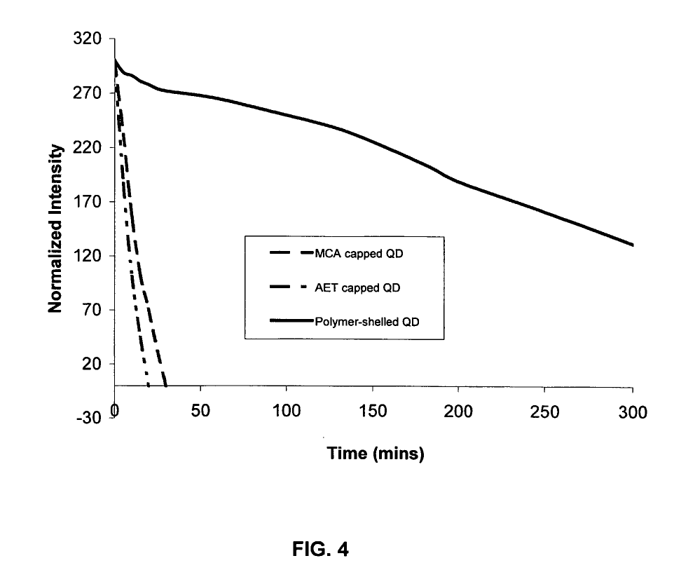

A scheme for carrying out coupling of the capping reagent with the polymer via the indirect contacting route is shown in FIG. 4. Firstly, nanocrystal cores may be prepared in coordination solvents such as trioctyl phosphine oxide (TOPO), resulting in the formation of a passivating layer on the nanocrystal core surface. Subsequently, the TOPO layer is displaced by the capping reagent. Displacement may occur by dispersion of TOPO-layered nanocrystals in a medium containing high concentrations of the capping reagent. This step is typically carried out either in an organic solvent or an aqueous solution. Preferred organic solvents include polar organic solvents such as pyridine, dimethylformamide (DMF), DMSO, dichloromethane, ether, chloroform, or tetrahydrofuran. Thereafter, the polymer to be coupled to the capping reagent may be prepared and added to the capped nanocrystal cores.

The method of the invention comprises, once the first layer of the water-soluble shell has been formed, the further step of coupling the nanocrystals capped with the capping reagent with a polymer having water-soluble groups. Coupling may be carried out in the presence of a coupling agent if desired. The coupling agent may be used to prime the capping reagent to render it reactive towards the polymer, or the coupling agent may be used to prime coupling moieties on the polymer to render them reactive towards the capping reagent. In a preferred embodiment, EDC (1-ethyl-3-[3-dimethylaminopropyl]carbodiimide) can be used as a coupling agents, optionally assisted by sulfoNHS (sulfo-N-hydroxysuccinimide). Other types of coupling reagents, including cross-linking agents, may also be used. Examples include, but are not limited to, carbodiimides such as diisopropylcarbodiimide, Carbodicyclohexylimide, N,N′-dicyclohexylcarbodiimide (DCC; Pierce), N-succinimidyl-S-acetyl-thioacetate (SATA), N-succinimidyl-3-(2-pyridyldithio)propionate (SPDP), ortho-phenylenedimaleimide (o-PDM), and sulfosuccinimidyl 4-(N-maleimidomethyl) cyclohexane-1-carboxylate (sulfo-SMCC) and azoles. The coupling agent catalyzes the formation of amide bonds between carboxylic acids and amines by activating the carboxyl group to form an O-urea derivative. This derivative reacts readily with the nucleophilic amine groups thereby accelerating the coupling reaction.

Equimolar quantities of coupling groups present in the capping reagent and of coupling moieties present in the polymer may be reacted. For illustration, assume that x moles of capping reagent having x moles of coupling groups can be attached to every 1 mole of nanocrystal cores. If y moles of polymer contain x moles of coupling moieties to completely react with 1 mole of nanocrystal cores (attached with x moles of capping reagent), then the mixing ratio of polymer to nanocrystal is at least y moles of polymer per mole of nanocrystal. In practice, capping reagents usually are reacted in excess to ensure complete capping on nanocrystals. Unreacted capping reagent can be removed via centrifugation, for example. The amount of polymer added to couple with the capped nanocrystal may be added in excess as well, typically in the region of about 10, or about 20 or about 30 to 1000 moles of polymer per mole of capped nanocrystal.

In order to couple the polymer to the capping reagent which is comprised on the surface of the nanocrystal core, the polymer is mixed with the capping reagent in the presence of a coupling agent. The coupling agent and the polymer may be added simultaneously to a solution containing the nanocrystal comprising the first layer (cf. Examples 1 and 2), or they may be added sequentially, the polymer being added after the coupling agent. The coupling agent acts as a initiator to activate the coupling groups and coupling moieties present in the capping reagent and the polymer, respectively. Thereafter, the polymer is coupled with the capping reagent to form a second layer that surrounds the nanocrystal core.

The coupling reaction can be carried out in an aqueous solution or in organic solvents. For example, the coupling reactions can be carried out in aqueous solutions, such as in water with suitable additives, including initiators, stabilizers or phase transfer reagents to improve the kinetics of the polymerization. It can also be carried out in a buffer solution, such as phosphate or ammonium buffer solution. In addition, the polymerization can be carried out in anhydrous organic solvents with suitable additives, such as coupling reagents and catalyst. Generally used organic solvents include DMF, DMSO, chloroform, dichloromethane, and THF.

Finally, once the second polymer layer of the organic shell has been formed, a last step may comprise reacting the polymer comprised in the second layer with a reagent suitable for exposing water soluble groups present in the second layer. For example, if the polymer used comprises an ester linkage (to protect carboxyl groups that may otherwise interfere in the formation of the second layer), the ester may be hydrolyzed by adding an alkaline solution (sodium hydroxide, for example) to the nanocrystal. So doing enables the carboxyl groups in the second layer to be released into the solution, that confers water solubility.

The present invention further refers to a nanocrystal, as disclosed herein, that is conjugated to a molecule having binding affinity for a given analyte. By conjugating the nanocrystal to a molecule having binding affinity for a given analyte, a marker compound or probe is formed. In such a probe, the nanocrystal of the invention serves as a label or tag which emits radiation, for example in the visible or near infrared range of the electromagnetic spectrum, that can be used for the detection of a given analyte.

In principle every analyte can be detected for which a specific binding partner exists that is able to at least somewhat specifically bind to the analyte. The analyte can be a chemical compound such as a drug (e.g. Aspirin® or Ribavirin), or a biochemical molecule such as a protein (for example, an antibody specific for troponin or a cell surface protein) or a nucleic acid molecule. When coupled to an appropriate molecule with binding affinity (which is also referred to as the analyte binding partner) for an analyte of interest, such as Ribavirin, the resulting probe can be used for example in a fluorescent immunoassay for monitoring the level of the drug in the plasma of a patient. In case of troponin, which is a marker protein for damage of the heart muscle, and thus in general for a heart attack, a conjugate containing an anti-troponin antibody and an inventive nanocrystal can be used in the diagnosis of heart attack. In case of an conjugate of the inventive nanocrystals with an antibody that it specific for a tumor associated cell surface protein, this conjugate may be used for tumor diagnosis or imaging. Another example is a conjugate of the nanocrystal with streptavidin.

The analyte can also be a complex biological structure including but not limited to a virus particle, a chromosome or a whole cell. For example, if the analyte binding partner is a lipid that attaches to a cell membrane, a conjugate comprising a nanocrystal of the invention linked to such a lipid can be used for detection and visualization of a whole cell. For purposes such as cell staining or cell imaging, a nanocrystal emitting visible light is preferably used. In accordance with this disclosure the analyte that is to be detected by use of a marker compound that comprises a nanoparticle of the invention conjugated to an analyte binding partner is preferably a biomolecule.

Therefore, in a further preferred embodiment, the molecule having binding affinity for the analyte is a protein, a peptide, a compound having features of an immunogenic hapten, a nucleic acid, a carbohydrate or an organic molecule. The protein employed as analyte binding partner can be, for example, an antibody, an antibody fragment, a ligand, avidin, streptavidin or an enzyme. Examples of organic molecules are compounds such as biotin, digoxigenin, serotronine, folate derivatives, antigens, peptides, proteins, nucleic acids and enzymes and the like. A nucleic acid may be selected from, but not limited to, a DNA, RNA or PNA molecule, a short oligonucleotide with 10 to 50 bp as well as longer nucleic acids.

When used for the detection of biomolecules a nanocrystal of the invention can be conjugated to the molecule having binding activity via surface exposed groups of the host molecule. For this purpose, a surface exposed functional group on the polymer such as an amine, hydroxyl or carboxylate group may be reacted with a linking agent. A linking agent as used herein, means any compound that is capable of linking a nanocrystal of the invention to a molecule having binding affinity for any biological target. Examples of the types of linking agents which may be used to conjugate a nanocrystal to the analyte binding partner are (bifunctional) linking agents such as ethyl-3-dimethylaminocarbodiimide or other suitable coupling compounds which are known to the person skilled in the art. Examples of suitable linking agents are N-(3-aminopropyl)3-mercapto-benzamide, 3-aminopropyl-trimethoxysilane, 3-mercaptopropyl-trimethoxysilane, 3-(trimethoxysilyl)propyl-maleimide, and 3-(trimethoxysilyl)propyl-hydrazide. The polymer coating may also be conjugated with a suitable linking agent that is coupled to the selected molecule having the intended binding affinity or analyte binding partner. For example, if the polymer coating comprises cyclodextrin moieties, then suitable linking agents may be used which may include, but is not limited to, ferrocene derivatives, adamantan compounds, polyoxyethylene compounds, aromatic compounds all of which have a suitable reactive group for forming a covalent bond with the molecule of interest.

Furthermore, the invention is also directed to a composition containing at least one type of nanocrystal as defined here. The nanocrystal may be incorporated into a plastic bead, a magnetic bead or a latex bead. Furthermore, a detection kit containing a nanocrystal as defined here is also part of the invention.

The invention is further illustrated by the following non-limiting examples and the attached drawings in which:

FIG. 1 depicts a generalized diagram of a water soluble nanocrystal of the invention (FIG. 1a), wherein FIG. 1b shows in greater detail the first layer that is attached to the surface of the nanocrystal core comprising amino ethylthiol as capping reagent, and polyacetyl acid polymer used for forming the second layer (cf. also FIG. 3). As can be seen from FIG. 1b, the nanocrystal comprises an interfacial region formed from the covalent bonding between at least one (neighboring) molecules of the coupling group of the capping reagent and one molecule of the coupling moiety of the polymer, such that the covalent bond between the coupling group on the capping reagent and the coupling moiety of the polymer serves as a bridge linking the capping reagent molecules together.

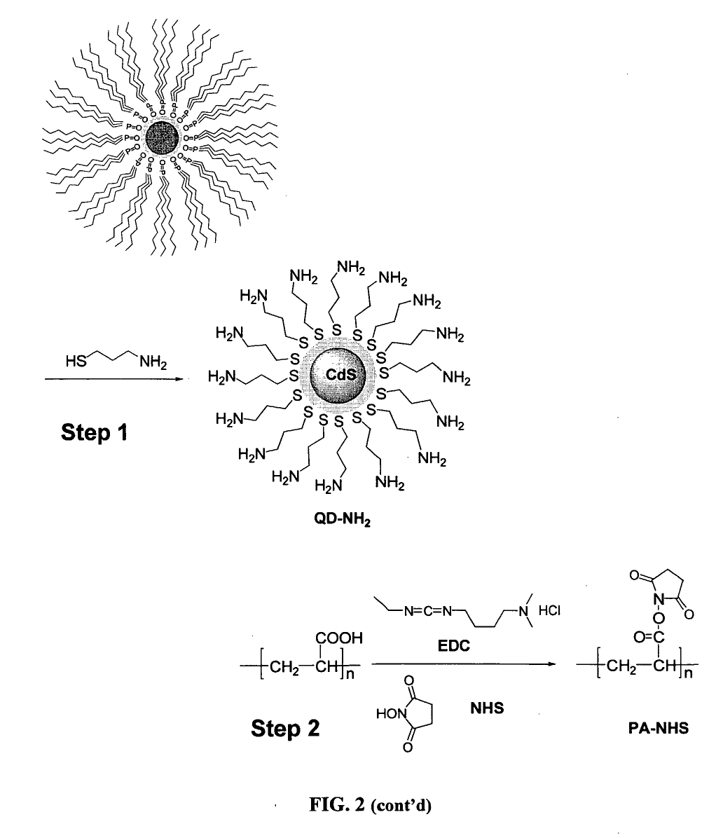

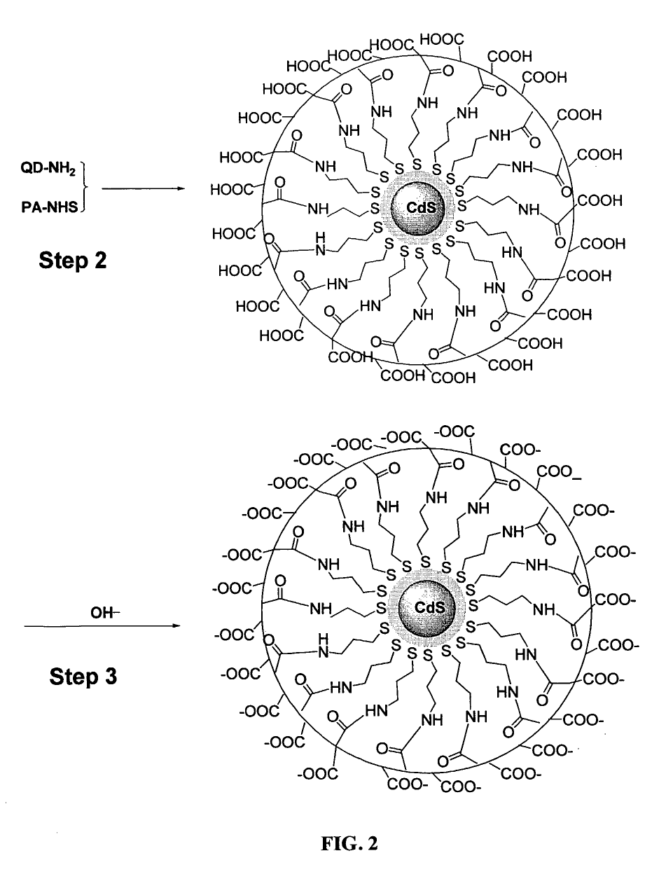

FIG. 2 shows a schematic diagram of a method for synthesizing a water soluble nanocrystal encapsulated in a polyamide polymer shell, formed via coupling using polyacetyl acid polymer to form the second layer of the shell. The capping reagent used is amino ethylthiol. In this example, the polyamide polymer shell also contains exposed carboxylic acid groups.

FIG. 3 shows a schematic diagram of a method for synthesizing a water soluble nanocrystal encapsulated in a polyamide polymer shell, formed via coupling using polyamine polymer to form the second layer of the shell. The capping reagent used is carboxyl ethylthiol. In this example, the polyamide polymer shell contains exposed amino groups.

FIG. 4 shows the stability of polymer shelled nanocrystals of the invention against chemical oxidation compared to the one of (CdSe)—ZnS core shell nanocrystals with were capped only with mercaptopropionic acid (MCA) or aminoethanethol (AET).

### EXAMPLE 1

### Preparation of Water-Soluble Nanocrystals with Coupled Polymers in Aqueous Solution

TOPO capped nanocrystals were first prepared in accordance with the following procedure.

Trioctylphosphine oxide (TOPO) (30 g) was placed in a flask and dried under vacuum (˜1 Torr) at 180° C. for 1 hour. The flask was then filled with nitrogen and heated to 350° C. In an inert atmosphere (dry box) the following injection solution was prepared: CdMe₂ (0.35 ml), 1 M trioctylphosphine-Se (TOPSe) solution (4.0 ml), and trioctylphosphine (TOP) (16 ml). The injection solution was thoroughly mixed, loaded into a syringe, and removed from the drybox.

The heat was removed from the reaction and the reaction mixture was transferred into vigorously stirring TOPO with a single continuous injection. Heating was resorted to the reaction flask and the temperature was gradually raised to 260-280° C. After the reaction, the reaction flask was allowed to cool to ˜60° C., and 20 ml of butanol were added to prevent solidification of the TOPO. Addition of large excess of methanol causes the particles to flocculate. The flocculate was separated from the supernatant liquid by centrifugation; the resulting powder can be dispersed in a variety of organic solvents to produce an optically clear solution.

A flask containing 5 g of TOPO was heated to 190° C. under vacuum for several hours then cooled to 60° C. after which 0.5 ml trioctylphosphine (TOP) was added. Roughly 0.1-0.4 μmols of CdSe dots dispersed in hexane were transferred into the reaction vessel via syringe and the solvent was pumped off. Diethyl zinc (ZnEt₂) and hexamethyidisilathiane ((TMS)₂S) were used as the Zn and S precursor, respectively. Equimolar amounts of the precursors were dissolved in 2-4 ml TOP inside an inert atmosphere glove box. The precursor solution was loaded into a syringe and transferred to an additional funnel attached to the reaction flask. After the addition was completed the mixture was cooled to 90° C. and left stirring for several hours. Butanol was added to the mixture to prevent the TOPO from solidifying upon cooling to room temperature.

TOPO coated quantum dots were then dissolved in chloroform, along with a large amount of aminoethylthiol (cf. FIG. 2, step 1). The mixture was ultrasonicated for 2 hours and then left at room temperature until the formation of precipitate was completed. The obtained solid was washed with chloroform several times and collected by centrifugation. Then the amino capped quantum dots were dissolved into a buffer solution with pH value of 8 and then added drop-wise into a solution of the poly(acrylic acid) polymer (Average Molecular Weight: 2,000 based on GPC), with EDC and sulfo-NHS present as coupling agents to activate the coupling groups on the capping reagent, and stirred at room temperature for 30 minutes (cf. FIG. 2, steps 2 and 3).

The reaction mixture was first stirred at 0° C. for 4 hours and then left to react at room temperature overnight. The obtained solution was dialyzed overnight and stored after degassing with nitrogen. Further purification was carried out by first washing the reaction solution with ether twice and centrifugation of the acidic (pH adjusted to about 4-5) polymer coated nanocrystal solution. The collected nanocrystals were then re-dissolved into water by adjustment of the pH value (to 7-8).

The physical-chemical properties of the polymer shell nanocrystals of the invention were compared to those of (CdSe)—ZnS core shell nanocrystals capped with only mercaptopropionic acid (MCA) or aminoethanethol (AET) as follows: To an aqueous solution of the nanocrystals, H₂O₂ was added in a final concentration of 0.15 mol/l and the chemical behaviour followed photospectroscopially (FIG. 4). For the nanocrystals that were coated only with MCA or AET oxidation of the nanocrystals was immediately detected and the nanocrystals precipitated within 30 minutes. In contrast, the shelled nanocrystals of the invention were significantly more stable against chemical oxidation which occurred only slowly.

### EXAMPLE 2

### Preparation of Water-Soluble Nanocrystals with Coupled Polymers in Organic Solution

TOPO capped nanocrystals were prepared in accordance with Example 1 and dissolved in chloroform, along with excess of 3-mercaptopropionic acid (cf. FIG. 4, step 1). The mixture was first sonicated for about 1 hour and then left at room temperature overnight until a large amount lot of precipitate was formed in the solution. The precipitate was collected by centrifugation and free 3-mercaptopropinoic acid was removed by washing with acetone for several times. The obtained 3-mercaptopropropionic acid capped quantum dots were dried briefly with argon gas and then dissolved into anhydrous DMF. To this solution, excess of EDC and NHS was added and then stirred at room temperature for about 30 minutes for activation and subsequent formation of the covalent coupling interface between the capping reagent and the polymer (cf. FIG. 4, step 2). From an additional funnel, polyethylenimine (Sigma-Aldrich Pte Ltd) with a molecular weight of 1200 (a MW of 400 to 60,000 is generally suitable), dissolved in anhydrous DMF was added dropwise with strong stirring. After the entire polyethylenimine solution was added, the reaction was continued at room temperature overnight for coupling of the polymer second layer to the capping reagent (cf. FIG. 4, step 3). Then, the DMF solvent was removed by rotary evaporation under reduced pressure and then dissolved into water. Further purification of the polymer coated quantum dots was carried out by washing with ether twice.

1. A water soluble nanocrystal comprising:
a nanocrystal core comprising at least one metal M1 selected from an element of subgroup Ib, subgroup IIb, subgroup IVb, subgroup Vb, subgroup VIb, subgroup VIIb, subgroup VIIb, main group II, main group III or main group IV of the periodic system of the elements (PSE), and
a water-soluble shell surrounding the nanocrystal core, said shell comprising:
a first layer comprising a capping reagent attached to the surface of the core of the nanocrystal, said capping reagent having at least one coupling group,
and a second layer comprising a polymer having at least one coupling moiety covalently coupled to the at least one coupling group of the capping reagent.

2. A water soluble nanocrystal comprising:
a nanocrystal core comprising at least one metal M1 selected from an element of main group II, subgroup VIIA, subgroup VIIIA, subgroup IB, subgroup IIB, main group III or main group IV of the periodic system of the elements (PSE), and at least one element A selected from main group V or main group VI of the PSE, and
a water-soluble shell surrounding the nanocrystal core, said shell comprising:
a first layer comprising a capping reagent attached to the surface of the core of the nanocrystal, said capping reagent having at least one coupling group,
and a second layer comprising a polymer having at least one coupling moiety covalently coupled to the at least one coupling group of the capping reagent.

3. The nanocrystal of claim 2, wherein the capping reagent comprises a terminal group having affinity for the surface of the core of the nanocrystal.

4. The nanocrystal of claim 2, wherein the capping reagent comprises at least one coupling group spaced apart from the terminal group by a hydrophobic region.

5. The nanocrystal of claim 4, wherein each coupling group comprises a functional group selected from amino, hydroxyl, carbonyl, carboxyl, nitrile, isocyanate and halide groups.

6. The nanocrystal of claim 2, wherein the capping reagent is a molecule having the formula (I): 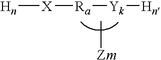
wherein
X is a terminal group selected from S, N, P, or O═P,
Rₐ is a moiety comprising at least 2 main chain carbon atoms,
Y is selected from N, C, —COO—, or —CH₂O—,
Z is a moiety comprising a polar functional group,
k is 0 or 1,
n is an integer from 0 to 3,
n′ is an integer from 0 to 2, wherein n′ is selected to satisfy the valence requirement of Y, and
m is an integer from 0 to 2.

7. The nanocrystal of claim 6, wherein the moiety Rₐ comprises 2 to 50 main chain atoms.

8. The nanocrystal of claim 6, wherein Rₐ is selected from the group consisting of alkyl, alkenyl, alkoxy and aryl moieties.

9. The nanocrystal of claim 8, wherein each of Rₐ is a moiety independently selected from the group consisting of ethyl, propyl, butyl, pentyl, cyclopentyl, cyclohexyl, cyclo-octyl, ethoxy, and benzyl.

10. The nanocrystal of claim 6, wherein Z is a functional group selected from the group consisting of amino, hydroxyl, carbonyl, carboxyl, nitrile, isocyanate and halide groups.

11. The nanocrystal of claim 10, wherein Z comprises 2 to 50 main chain atoms.

12. The nanocrystal of claim 11, wherein Z further comprises an amide or an ester linkage.

13. The nanocrystal of claim 2, wherein the capping reagent is a compound selected from the group consisting of:

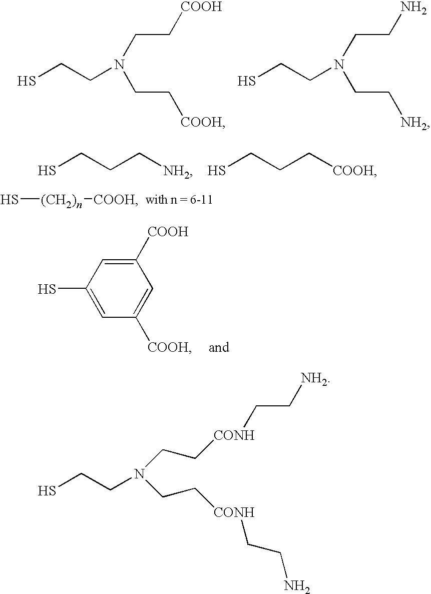

14. The nanocrystal of claim 4, wherein the coupling group of the capping reagent comprises a polymerizable unsaturated carbon-carbon bond.

15. The nanocrystal of claim 14, wherein the capping reagent is selected from the group consisting of ω-thiol terminated methyl methacrylate, 2-butenethiol, (E)-2-Butene-1-thiol, S-(E)-2-butenyl thioacetate, S-3-methylbutenyl thioacetate, 2-quinolinemethanethiol, and S-2-quinolinemethyl thioacetate

16. The nanocrystal of claim 2, wherein the polymer has the formula (III): 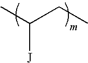
wherein
J is a coupling moiety that is reactive towards the at least one coupling group of the capping reagent, and
m is an integer of at least 1.

17. The nanocrystal of claim 2, wherein the polymer comprises at least two coupling moieties that are reactive towards the at least one coupling group of the capping reagent.

18. The nanocrystal of claim 17, wherein the polymer has the formula (IV): 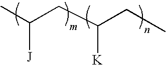
wherein
J and K are coupling moieties, said J and K are the same or different, and
each of m and n is an integer of at least 1.

19. The nanocrystal of claim 2, wherein the polymer comprises at least three coupling moieties that are reactive towards the at least one coupling group of the capping reagent.

20. The nanocrystal of claim 19, said polymer having the formula (V): 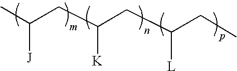
wherein
J, K and L are coupling moieties, said J, K and L are the same or different, and
each of m, n and p is an integer of at least 1.

21. The nanocrystal of claim 16, wherein at least one of said coupling moieties J, K or L comprises a hydrophilic group which confers water solubility to the water-soluble shell.

22. The nanocrystal of claim 17, wherein the polymer further comprises at least one moiety having a hydrophilic group that confers water solubility to water-soluble shell.

23. The nanocrystal of claim 17, wherein said coupling moieties J, K and L each comprises a functional group selected from amino, hydroxyl, carbonyl, carboxyl, nitrile, isocyanate and halide groups.

24. The nanocrystal of claim 23, wherein the coupling moieties of the polymer are homofunctional.

25. The nanocrystal of claim 24, wherein the polymer is selected from the group consisting of polyamine, polycarboxylic acid, and polyvinyl alcohol.

26. The nanocrystal of claim 18, wherein the polymer comprises a diblock copolymer.

27. The nanocrystal of claim 26, wherein said diblock copolymer is selected from the group consisting of poly(acrylic acid-b-methyl methacrylate), poly(methyl methacrylate-b-sodium acrylate), poly(t-butyl methacrylate-b-ethylene oxide), poly(methyl methacrylate-b-sodium methacrylate), and poly(methyl methacrylate-b-N,N-dimethyl acrylamide).

28. The nanocrystal of claim 14, wherein the polymer comprises poly(acetylene), polyacrylic acid, and polyethylenimine.

29. The nanocrystal of claims 2, wherein the molecular weight of the polymer is between about 2000 to about 750000.

30. The nanocrystal of claim 2, wherein the nanocrystal is a core-shell nanocrystal.

31. The nanocrystal of claim 30, wherein the metal is selected from the group consisting of Zn, Cd, Hg, Mn, Fe, Co, Ni, Cu, Ag, and Au.

32. The nanocrystal of claim 30, wherein the element A is selected from the group consisting of S, Se, and Te.

33. The nanocrystal of claim 32, wherein the nanocrystal is a core shell nanocrystal selected from the group consisting of CdS, CdSe, MgTe, CdTe, ZnS, ZnSe, ZnTe, HgS, HgSe, and HgTe.

34-40. (canceled)

41. The nanocrystal of claim 2, further comprising a molecule having binding affinity for a given analyte being conjugated to the second layer of the polymer shell.

42-43. (canceled)

44. A method of detecting an analyte using a nanocrystal as defined in claim 2.

45. A method of preparing a water soluble nanocrystal comprising:
providing a nanocrystal core comprising at least one metal M1 selected from an element of subgroup Ib, subgroup IIb, subgroup IVb, subgroup Vb, subgroup VIb, subgroup VIIb, subgroup VIIIb, main group II, main group III or main group IV of the periodic system of the elements (PSE),
reacting the nanocrystal core with a capping reagent, thereby attaching the capping reagent to the surface of the nanocrystal core and forming a first layer surrounding the nanocrystal core,
and
coupling the capping reagent with a polymer having at least one coupling moiety that is reactive towards the at least one coupling group of the capping reagent, thereby forming a second layer covalently coupled to the first layer and completing the formation of a water soluble shell surrounding the nanocrystal core.

46. A method of preparing a water soluble nanocrystal comprising:
providing a nanocrystal core comprising at least one metal M1 selected from the group consisting of an element of subgroup IIB-VIB, IIIB-VB or IVB, main group II or main group III of the periodic system of the elements (PSE), and at least one element A selected from an element of the main group V or VI of the periodic system of the elements,
reacting the nanocrystal core with a capping reagent, thereby attaching the capping reagent to the surface of the nanocrystal core and forming a first layer surrounding the nanocrystal core,
and
coupling the capping reagent with a polymer having at least one coupling moiety that is reactive towards the at least one coupling group of the capping reagent, thereby forming a second layer covalently coupled to the first layer and completing the formation of a water soluble shell surrounding the nanocrystal core.

47. The method of claim 46, wherein the capping reagent is hydrophilic.

48. The method of claim 46, wherein the capping reagent is hydrophobic.

49. The method of claim 46, wherein each coupling group present in the capping reagent comprises a functional group selected from amino, hydroxyl, carbonyl, carboxyl, nitrile, isocyanate and halide groups.

50. The method of claims 46, wherein the capping reagent has the formula (I): 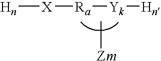
wherein
X is a terminal group selected from S, N, P, or O═P,
Rₐ is a moiety comprising at least 2 main chain carbon atoms,
Y is selected from N, C, —COO—, or —CH₂O—,
Z is a moiety comprising a polar functional group,
k is 0 or 1,
n is an integer from 0 to 3,
n′ is an integer from 0 to 2, wherein n′ is selected to satisfy the valence requirement of Y, and
m is an integer from 0 to 2.

51. The method of claim 46, wherein the capping reagent is a compound selected from the group consisting of

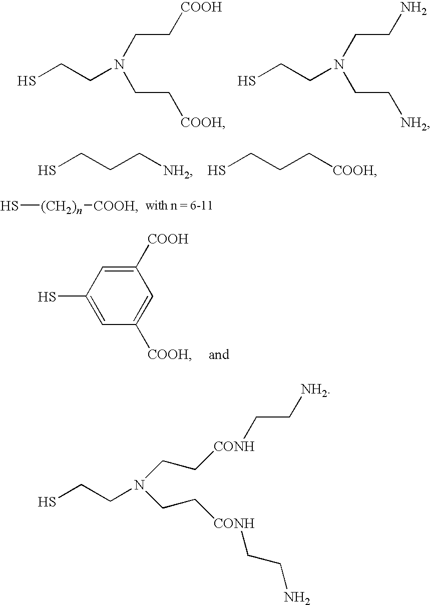

52. The method of claim 46, further comprising the step of activating coupling groups of the capping reagent before coupling the capping reagent to the polymer.

53. The method of claim 52, wherein the step of activating comprises reacting the nanocrystal comprising the first layer of capping reagent with a coupling agent.

54. The method of claim 53, wherein the coupling agent is selected from the group consisting of 1-ethyl-3-[3-dimethylaminopropyl]carbodiimide (EDC), sulfo-N-hydroxysuccinimide, N,N′-Dicyclohexylcarbodiimide (DCC), N,N′-dicyclohexyl carbodiimide, N-(3-dimethylaminopropyl)-N′-ethylcarbodiimide, and N-hydroxysuccinimide.

55. The method of claim 53, wherein coupling the capping reagent with a polymer comprises adding the polymer and the coupling agent together to a solution containing the nanocrystal comprising the first layer.

56. The method of claim 46, wherein the coupling is carried out in an aqueous buffer solution.

57. The method of claim 56, wherein the aqueous buffer solution comprises a phosphate or ammonium buffer solution.

58. The method of claim 46, wherein the coupling is carried out in a polar organic solvent.

59. The method of claim 58, wherein the organic solvent is selected from the group consisting of pyridine, DMF, and chloroform.

60. The method of claim 46, wherein the polymer has the formula (III): 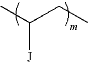
wherein
J is a coupling moiety that is reactive towards the at least one coupling group of the capping reagent, and
m is an integer of at least 1.

61. The method of claim 46,
wherein the polymer has the formula (IV): 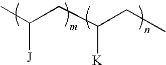
wherein
J and K are coupling moieties, said J and K are the same or different, and
each of m and n is an integer of at least 1.

62. The method of claims 46,
wherein the polymer has the formula (IV): 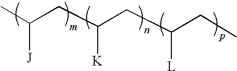
wherein
J, K and L are coupling moieties, said J, K and L are the same or different, and
each of m, n and p is an integer of at least 1.

63. The method of claim 46, further comprising reacting the polymer comprised in the second layer with a reagent suitable for exposing water soluble groups present in the second layer.
